SEQUENCE LISTING (9 sequences)

SEQ ID NO: 1 - DNA, 1155 bp, Brassica juncea
  exon (1)..(1155)
atgggtgcaggtggaagaatgcaagtgtctcctccctcgaagaagtctgaaaccgacaccatcaagagggtaccctgcgagacaccgcccttcactgtcggagaattgaagaaagcaatcccaccgcactgtttcaaacgttcgatccctcgttctttctcctacctaatctgggacatcatcatagcctcctgcttctactacgtcgccaccacttacttccctctactacctcaccctctatcctacttcgcctggcctttgtactgggcctgccagggctgcgtcctaaccggcgtctgggtcatagcccacgagtgcggccaccacgccttcagcgactaccagtggcttgacgacaccgtcggtctaatcttccactccttcctactagtcccttacttctcctggaagtacagtcatcgaaggcaccattccaacactggctccttggagagagacgaagtgtttgtccccaagaagaagtcagacatcaagtggtacggcaagtacttgaacaaccctttgggacgcaccgtgatgttaacggttcagttcactttgggctggcctttgtacttagccttcaacgtctcgggaagaccttacgacggcggcttcgcttgccatttccaccctaacgctcccatctacaacgaccgcgagcgtttgcagatatacatctccgacgctggcatcttggccgtctgctacggtctattccgttacgctgctgcccaaggagttgcctcgatggtctgcttctacggagtcccgcttctgatagtcaacgggttgttagttttgatcacttacttgcagcacacgcatccttccctgcctcactacgattcgtctgagtgggattggttgaggggagcgttggctaccgttgacagagactacgggatcttgaacaaggtcttccacaatatcacggacacgcacgtggcgcatcacctgttctcgaccatgccgcattatcacgcgatggaagctaccaaggcgataaagccgatactgggagagtattatcagttcgatgggacgccggtggttaaggcgatgtggagggaggcgaaggagtgtatctatgtggaaccggacaggcaaggtgagaagaaaggtgtgttctggtacaacaataagttatag

SEQ ID NO: 2 - DNA, 1152 bp, Brassica juncea
  CDS (1)..(1152)
  exon (1)..(1152)
atggttgttgctatggaccagcgcaccaatgtgaacggagatgccggtgcccggaaggaagaagggtttgatccgagcgcacaaccgccgtttaagatcggggacataagggctgcgattcctaagcattgttgggtgaaaagtcctttgagatctatgagctacgtagccagagacatttgtgccgtcgcggctttggccattgccgccgtgcattttgatagctggttcctctgtcctctctattgggtcgcccaaggaacccttttctgggccatcttcgtcctcggccacgactgtggacacgggagtttctcagacattcctctgctgaatagtgtggttggccgtattcttcattccttcatcctcgttccttaccatggttggagaataagccatcggacacaccaccagaaccatggccatgttgaaaacgacgagtcttgggttccgttaccagaaaggttatacaagaatttaccccacagtactcggatgctcagatacactgtccctctgcccatgctcgcttacccgatctatctgtggtacagaagtcctggaaaagaagggtcacattttaacccatacagtggtttatttgctccaagcgagagaaagcttattgcaacttcgactacttgctggtccataatgttggcaattcttatctgtctttccttcctcgttggtccagtcacagttctcaaagtatacggtgttccttacattatctttgtgatgtggctggacgctgtcacttacttgcatcaccatggtcatgatgagaagttgccttggtacagaggcgaggaatggagttacttacgtggaggattaacaactattgatagagattacggaattttcaacaacattcatcacgacattggaactcacgtgatccatcatcttttcccacaaatccctcactatcacttggtcgatgctacaaaagcagctaaacatgtgttgggaagatactacagagaaccaaagacgtcaggagcaataccgatccacttggtggagagtttagcagcaagtattaagaaagatcattacgtcagtgacactggtgacattgtcttctacgggactgatccagatctctacgtttatgcttctgacaaatctaaaatcaattaa

SEQ ID NO: 3 - DNA, 1152 bp, Brassica juncea
  CDS (1)..(1152)
  exon (1)..(1152)
atggttgttgctatggaccagcgcaccaatgtgaacggagatgccggtgcccggaaggaagaagggtttgatccgagcgcacaaccgccgtttaagatcggggacataagggctgcgattcctaagcattgttgggtgaaaagtcctttgagatctatgagctacgtaaccagagacatttttgccgtcgcggctttggccatggccgccgtgcattttgatagctggttcctttggcctctttattgggtcgcccaaggaacccttttctgggccatcttcgtcttgggccacgactgtggacacgggagtttctcagacattcctctgctgaatagtgtggttggccatattcttcattccttcatcttggttccttaccatggttggagaataagccatcggacacaccaccagaaccatggccatgttgaaaacgacgagtcttgggttccgttaccagaaaggttatacaagaatttaccccacagtactcggatgttgagatacactgtccctctgcccatgttggcttacccgatctatctgtggtacagaagtcctggaaaagaagggtcacattttaacccatacagttctttatttgctccaagcgagagaaagcttattgcaacttcgactacttgctggtccataatgttggcaactcttgtctatctttccttccttgttgatccagtcacagttcttaaagtatacggtgttccttacattatctttgtgatgtggctggacgctgtcacttacttgcatcaccatggtcatgatgagaagttgccttggtacagaggcgaggaatggagttacttacgtggaggattaacaactattgatagagattacggaattttcaacaacattcatcacgacattggaactcacgtgatccatcatcttttcccacaaatccctcactatcacttggtcgatgctacaaaagcagctaaacatgtgttgggaagatactacagagaaccaaagacgtcaggagcaataccgatccacttggtggagagtttagttgcaagtattaagaaagatcattacgtcagtgacactggtgacattgtcttctacgagactgatccagatctttacgtttatgcttctgacaaatctaaaatcaattaa

SEQ ID NO: 4 - DNA, 1419 bp, Thraustochytrid
  CDS (1)..(1419)
  exon (1)..(1419)
atgatctggcgggaggaatttggaaaggcagtagcacgtccgttagaaccagaagtgtacgcacgcaaacgcgagcagctcggacataagaagttctcctgggatgagataaatcaacataccaagcgtgacgatctatggatcgttgtcgagggcaaggtgtttgatgtgacccctttcgtagaacgccaccctggtggctggcgtccaattacgcacagtagtggtaaagacggaacagatgcatttagtgaatttcaccccgctagcgtcttggaacgttggatgcctcagtactacatcggtgacgtggacaagtatgaggtttctgccttggtccgcgactttagagccatcaaacaagaactcttggctcgtgggtattttgaaaacaccacctcctattactatgcaaagtacatctggtgcgcttccatgttcgcgccagctctgtatggagtgttgtgctgcacgtcaacatttgcgcatatgctatccgctattggaatggctatgttttggcaacaaatagcttttattggtcatgatgctggccacaacgctgtatctcatgttcgcgatatggatctcttttgggcaggttttatcggtgatatgcttggtggagtggggcttagctggtggaagctgtcccacaacactcaccactgtgtgacaaacagtgtcgagaatgacccagacatccaacacttgccttttctggccattacaaataagctcttcaaacgcttctacagtacattccatgatcgatactttgaggcagatatctttgctcgcttctttgtaggttaccaacacattctgtactatccggtgatgatggttgcacgcttcaatctgattcttcaaagctggctcacccttctttctcgtgaacgtattgactaccgttactcggagatgcttgctcttgctattttctgggtgtggttctataagtttgtcatgtgcttgccgtacaatgagcgtattccatatgttgtgctctcttacgcagttgctggcatcctccatgtccagatctgtatttctcactttatgatggaaactttccacggtcgctctaccgaggaatggattcgtcatcagctgcggacatgtcaggatgtaacatgtccgttttacatggattggtttcatggcggtttgcaatttcagactgagcatcacatgtggccccgcttgccccgtaggaatcttcgggtggcacgtgctcgtctgattgagctctgtgcaaaatacaacctcaattatgttgaaatggactttattgaatcaaacaagcaccttatcagatgcctgcgtaagactgccatggaagcacgtaaactcaagtctggagatgctggattttatgaaagtccaatgtgggaaagtctcaacctccgtggttga

SEQ ID NO: 5 - DNA, 1419 bp, Thraustochytrid
  CDS (1)..(1419)
  exon (1)..(1419)
atgatatggcgagaagagttcggtaaggctgttgcccgtcctttggaacctgaggtctatgcgcgaaaaagagaacaactcggtcacaagaaattttcttgggacgagataaatcagcatactaaaagggatgatttgtggatagttgtcgaaggtaaagtttttgatgttactcctttcgtggagagacatccgggtgggtggcgacctataacccattcctccggcaaagatggtaccgatgcctttagtgaatttcatccggcctcggtcctagagcgttggatgccgcaatattatattggtgatgtggataaatacgaagtgtcggcattagtaagagacttccgtgccataaagcaagaactacttgcccgtggttattttgaaaacacaacgtcatactattacgctaagtatatttggtgtgcatctatgttcgctcctgcgttatatggagtattgtgttgtacctccacattcgcacatatgctatcagcaataggaatggcaatgttctggcaacaaatcgctttcatagggcatgacgcagggcataatgcagtttcgcatgttagggacatggatcttttttgggccggctttataggagatatgttagggggtgtgggtttgtcatggtggaagttgtctcataatacccatcactgtgtcactaactctgtggaaaatgatcctgatatccaacacttgccgttcctcgcaataactaacaaattattcaaaagattttatagtacatttcatgataggtatttcgaagccgatatctttgccaggtttttcgttggataccaacatatcttatattatccagtaatgatggtggctcgcttcaatttgatcttacagtcttggttgaccttgttaagtcgagaaagaatagattatagatactctgaaatgttagccctggccatattctgggtctggttctataaatttgtgatgtgtctaccatacaatgagagaataccttatgttgttttatcctatgcggtagcaggaatattgcatgtacaaatatgtatatcccattttatgatggaaactttccatggtcgttcaaccgaggaatggatacgccaccaacttaggacctgccaagatgtgacatgtcccttctatatggattggtttcacggaggtttgcagtttcaaactgagcatcacatgtggccacgccttcccagaaggaacctgagagttgccagagcccgactaatcgaattatgcgctaaatacaatttgaattacgtcgaaatggattttatcgaatcaaacaaacaccttattcgttgccttaggaaaactgctatggaggctcgtaagttgaagtcaggcgatgccggtttctatgagtccccaatgtgggaatctctgaacctgagaggttga

SEQ ID NO: 6 - DNA, 1122 bp, Thraustochytrid
  CDS (1)..(1122)
  exon (1)..(1122)
atgaaggagatgaactcaggagtcgttcgtcgtgccatcaaattcggcactcaagatattaacgatgcatgcggtgtagtcgccgtaggcaccaacgagcttattggcgagtctggccctaaatcccaggccgacgaggccaagaagcaggaccgtcgcaagcgtcaggctcttggaacccacatgttcaccgcatatcttttggtgtacgctgcccttatgattgtttctgcctttgaccttctccctgtgatggattgggaggtcatgaagtttgacactgctgaggtcgtttccgtatggctccgtacccacatgtgggtgcccttcctgctctgcttcatctaccttgtagttatcttcgggattcagtactacatggaggacaaggctgagtttgatcttcgtaagccgcttgctgcctggagcgcatttcttgccatcttcagtgttggtgcttccatccgcactgtgcctgtcctcctcaagatgctctacgagaagggaactcatcacgtgctttgtggtgacacccgccaggactgggtaattgataacccggctggagtgtggacaatggccttcatcttttccaagatccctgagcttattgacaccctcttccattgtgctccgcaagcgcaagctcattactctccactggtaccaccacgtccaccgtgcttctcttctgctggcacgcctgggcccacttttgcccttaccggcattgtcttcgccgccatcaatgcttcagtgcatgctatcatgtacgcgtactatgcttacaccgctctcggataccgcccgactgcctatgcaatctacattactctgattcagattgcacagatggttgttggcactgctgttaccttctacattggctacgacatggcctttgttacccctcagccgttccgtcttgatatgaagctcaactgggacccgcttgacaagaacatcaacactgagccttcttgcaagggcgccaactcctccaatgctatctttggtgtgatcatgtacgcctcgtacttgtacctcttttgcctcttcttctacatggcgtaccttcgccccaagaccaagaagacgacggccgctaagaagaccgattaa

SEQ ID NO: 7 - DNA, 1419 bp, thraustochytrid; identical to SEQ ID NO: 5
  CDS (1)..(1419)
  exon (1)..(1419)

SEQ ID NO: 8 - DNA, 1403 bp, Phaeodactylum tricornatum
  exon (1)..(1403)
atgatggaaacaaataatgaaaataaagaaaaattaaaattatatacttgggatgaagtatcaaaacataatcaaaaaaatgatttatggattatagttgatggtaaagtttataatattacaaaatgggtaccattacatccaggtggtgaagatatattattattatcagcaggtagagatgcaacaaatttatttgaaagttatcatccaatgacggataaacactattccttaattaaacaatatgaaattggatatatatcatcatatgaacatccaaaatatgttgaaaaaagtgaattctatctacattgaaacaacgtgttagaaaacatttccaaacttcatcacaagatccaaaagtttcagttggagttttcacaagaatggtgttaatttatttattcctatttgttacttactatttatcacaattctctacggatagattttggttaaattgtatattcgctgttttatatggtgttgcaaattcgttatttggattacacacgatgcatgacgcttgccacacagcaatcactcataatccaatgacttggaaaatattgggtgcaacatttgatttgttcgctggtgcttcattctatgcatggtgtcatcaacatgtgattgggcatcatttatatacaaatgtaagaaatgcagatccagacttgggtcaaggtgaaattgattttcgtgttgttacaccatatcaagcaagatcatggtaccataaatatcaacatatttacgcaccaattctatatggagtttacgctttaaaatatcgtattcaagatcacgaaatctttacaaagaaatcaaatggtgcaattagatattcaccaatatcaacgattgatactgcaattttcatacttggtaaattggttttcattatctctcgtttcatactcccattaatctataatcattcattctctcatttaatttgtttcttcctaatctctgaattggttttaggttggtatttagccatttcttttcaagttagtcatgtagttgaagatcttcaattcatggcaacacctgaaattttcgatggtgctgatcacccattaccaacaaccttcaatcaagattgggcaattcttcaagttaaaactactcaagattatgctcaagattcagttttaagtactttcttttctggtggtttaaatttacaagttattcatcattgtttcccaacaattgctcaagattattacccacaaattgttccaattcttaaagaagtttgtaaagaatataatgttacatatcattataagccaacatttactgaagcaataaagtctcatatcaactatctttacaaaatgggtaatgatccagactatgtcagaaaaccagtaaacaaaaacgattaa

SEQ ID NO: 9 - DNA, 1560 bp, Thraustochytrid
  MISC_FEATURE (462)..(462): Xaa may be any naturally-occurring amino acid
atgacggtcggctacgacgaggagatcccgttcgagcaggtccgcgcgcacaacaagccggatgacgcctggtgcgcgatccacgggcacgtgtacgatgtgaccaagttcgcgagcgtgcacccgggcggcgacattatcctgctggccgcaggcaaggaggccaccgtgctgtacgagacttaccatgtgcggggcgtctcggacgcggtgctgcgcaagtaccgcatcggcaagctgccggacggccaaggcggcgcgaacgagaaggaaaagcggacgctctcgggcctctcgtcggcctcgtactacacgtggaacagcgacttttacagggtaatgcgcgagcgcgtcgtggctcggctcaaggagcgcggcaaggcccgccgcggaggctacgagctctggatcaaggcgttcctgctgctcgtcggcttctggagctcgctgtactggatgtgcacgctggacccctcgttcggggccatcctggccgccatgtcgctgggcgtctttgccgcctttgtgggcacgtgcatccagcacgacggcaaccacggcgcctttgcccagtcgcgatgggtcaacaaggttgccgggtggacgctcgacatgatcggcgccagcggcatgacgtgggagttccagcacgtcctgggccaccatccgtacacgaacctgatcgaggaggagaacggcctgcaaaaggtgagcggcaagaagatggacaccaagctggccgaccaggagagcgatccggacgtcttttccacgtacccgatgatgcgcctgcacccgtggcaccagaagcgctggtaccaccgtttccagcacatttacggccccttcatctttggcttcatgaccatcaacaaggtggtcacgcaggacgtcggtgtggtgctccgcaagcggctcttccagattgacgccgagtgccggtacgcgagcccaatgtacgtggcgcgtttctggatcatgaaggcgctcacggtgctctacatggtggccctgccgtgctacatgcagggcccgtggcacggcctcaagctgttcgcgatcgcgcactttacgtgcggcgaggtgctcgcaaccatgttcattgtgaaccacatcatcgagggcgtctcgtacgcttccaaggacgcggtcaagggcacgatggcgccgccgaagacgatgcacggcgtgacgcccatgaacaacacgcgcaaggaggtggaggcggaggcgtccaagtctggcgccgtggtcaagtcagtcccgctcgacgactgggccgtcgtccagtgccagacctcggtgaactggagcgtcggctcgtggttctggaatcacttttccggcggcctcaaccaccagattgagcaccacctgttccccggrctcagccacgagacgtactaccacattcaggacgtctttcagtccacctgcgccgagtacggcgtcccgtaccagcacgagccttcgctctggaccgcgtactggaagatgctcgagcacctccgtcagctcggcaatgaggagacccacgagtcctggcagcgcgctgcctga

(19) United States
(12) Patent Application Publication
(10) Pub. No.: US 2010/0120103 A1
(43) Pub. Date: May 13, 2010

(54) Recombinant Production Docosahexaenoic Acid (DHA) in Yeast

(75) Inventors: Villoo Morawala Patell, Karnataka (IN); K.R. Rajyashri, Karnataka (IN)

Correspondence Address: SALIWANCHIK LLOYD & SALIWANCHIK;A PROFESSIONAL ASSOCIATION, PO Box 142950, GAINESVILLE, FL, 32614, US

(73) Assignee: AVESTHAGEN LIMITED, Bangalore (IN)

(21) Appl. No.: 11/721,660

(22) PCT Filed: Nov. 9, 2005
(86) PCT No.: PCT/IB2005/003441

§ 371 Date: Jun. 13, 2007

(30) Foreign Application Priority Data
Dec. 14, 2004 (IN) 1372/CHE/2004

(51) Int. Cl. C12P 7/64; C12N 1/16; C12N 1/19

(52) U.S. Cl. 435/134; 435/255.1; 435/255.2

(57) ABSTRACT

The present invention relates to a specifically novel recombinant method of production of the omega-3 fatty acid, Docosahexaenoic acid by a potentially safe recombinant organism Saccharomyces cerevisiae. The invention describes the process of bioconversion of oleic acid to docosahexaenoic acid through a series of enzymatic conversions facilitated through the cloning of the respective genes into appropriate vectors and the final expression of the DHA in the host cell, Yeast.

### FIELD OF INVENTION

The present invention describes the pathway engineering of Yeast for the conversion of oleic acid normally synthesized in yeast, to DHA by introducing 5 desaturases and elongases isolated from appropriate sources. It also includes cloning the respective genes into appropriate vectors and introduces them into yeast for the production of DHA in yeast.

### BACKGROUND OF INVENTION

Docosahexaenoic acid (DHA) (22:6) is a omega-3-fatty acid, so called because it has a double-bond 3 carbon atoms away from the methyl end of the molecule. All the fatty acids which are essential in the human diet are either omega-3 or omega-6. Although DHA can be synthesized in the body from alpha-linolenic acid (a simpler omega-3 found in the linseed oil and perilla oil), the capacity for the synthesis declines with age. The omega-3 and omega-6 family of fatty acids are essential because they cannot be synthesized in the body, but must be obtained in the diet. Fatty acids are contained in the membranes of every cell in the body, but essential fatty acids are particularly concentrated in the membranes of the brain cells, heart cells and the immune system cells.

DHA is an essential component of the brain and the retina and is implicated in a number of other essential body functions. It is especially important for the growth and the development of the fetal and the neonatal brain. Reduced levels of DHA during this period lead to the retarded neural development, visual acuity and reduced childhood intelligence. Postnatal deficiency of DHA may also induce a predisposition to adult degenerative diseases, while supplementary intake of DHA in the diet has been documented to have positive effect on the heart—it lowers LDL levels and triglycerides—and has positive effects. It is also used in the treatment of rheumatoid arthritis.

DHA deficiencies are associated with fetal alcohol syndrome, attention deficit hyperactivity disorder, cystic fibrosis, phenylketonuria, unipolar depression, aggressive hostility and adrenoleukodystrophy. Decreases of DHA in the brain are also associated with cognitive decline during aging and with onset of sporadic Alzheimer's disease.

The leading cause of death in the western nations is cardiovascular diseases. Epidemiological studies have shown a strong correlation between fish consumption and reduction in the sudden death from myocardial infarction. DHA is the active component in fish. Although most fish oils are high in EPA and DHA, there are some fish oils which are not. Flounder, swordfish and sole are particularly low in EPA and DHA. Fish oils with the highest levels of EPA and DHA include mackerel, herring and salmon. Some fish, such as cod and haddock, store most of their fat in the liver, therefore the liver oils of these should be taken than the oil from the fillet. Not only does fish oil reduce triglycerides in the blood and decrease thrombosis, but it also prevents cardiac arrhythmia. DHA purified from fish oil has been shown to lower the blood pressure and reduce the blood viscosity. The evidence indicates that DHA increases the red blood cell membrane fluidity, thereby increasing the deformability of the blood cells so they can move through capillaries more easily and thereby lower blood viscosity and blood pressure. DHA may also reduce blood pressure by lowering cortisol. The association of DHA deficiency with depression is the reason for the robust positive correlation between depression and myocardial infarction. DHA also has a positive effect on diseases such as hypertension, arthritis, artherosclerosis, adult onset diabetes mellitus, thrombosis and some cancers. The most dramatic effects of fish oil on the heart, however, are in connection with cardiac arrhythmias (irregular heartbeats). In the United States, a quarter of a million people die annually within an hour of a heart attack as a result of arrhythmia. Brain development is a complex interactive process in which early disruptive events can have long lasting effects on functional adaptations. Long chain polyunsaturated fatty acids (LCPUFA), specifically arachidonic acid and Docosahexaenoic acid accrue rapidly in the gray matter of the brain during development and brain fatty acid (FA) composition reflects dietary availability. Dietary n-3 fatty acid deficiency influences specific neurotransmitter systems, particularly the dopamine systems of the frontal cortex. Most of the dry weight of the brain is lipid (fat) because brain activity depends greatly upon the function provided by lipid membranes. Compared to other body tissues, brain of DHA and arachidonic acid is very high. DHA is particularly concentrated in the membranes that are functionally active, namely synapses and in the retina. The greatest dependance on dietary DHA occurs in the fetus during the last third week of pregnancy and (to a lesser extent) in the infant during the first 3 months after birth. It is during this period that brain synapses are forming most rapidly, and an infant's demand for DHA exceeds the capacity of the enzymes to synthesize it.

An important role for Docosahexaenoic acid (DHA) within the retina is suggested by its high levels and active conservation in this tissue. Animals raised on n-3-deficient diets have large reductions in retinal DHA levels that are associated with altered retinal function as assessed by electroretinogram. Role of DHA in retinal function is significant particularly within the rod photoreceptor outer segments where DHA is found at its highest concentration. Neonatal dietary supply of DHA is required for the normal development of the retinal function.

Animal experiments and clinical intervention studies indicate that omega-3 fatty acids have anti-inflammatory properties and, therefore, might be useful in the management of inflammatory and autoimmune diseases. Coronary heart disease, major depression, aging and cancer are characterized by a high level of interleukin 1 (IL-1), a proinflammatory leukotrine LTB-4 produced by a omega-6 fatty acids. There have been a number of clinical trials assessing the benefits of dietary supplementation with fish oils in several inflammatory and autoimmune diseases in humans, including rheumatoid arthritis, Crohn's disease, ulcerative colitis, psoriasis, multiple sclerosis and migraine headaches.

The n-3 polyunsaturated fatty acids (PUFA) eicosapentanoic acid (EPA) and docosahexaenoic acid (DHA) are found in high proportions in oily fish and fish oils. Vegetarian diets are relatively low in alpha-linolenic acid (ALA) compared to linoleic acid (LA) and provide little, if any, eicosapentanoic acid (EPA) and Docosahexaenoic acid. Hence, vegetarians need to make dietary changes to optimize the requirements of DHA.

The inconsistency in the DHA content of fish oils and the undesirable fish odor of the product has spurred research for alternate sources of DHA. A group of marine protists, the thraustochytrids, are natural producers of ω-3 fatty acids including DHA. These organisms have, in the recent past, been cultured and used for the production of DHA. However, cost effective alternatives to be explored for fulfilling the needs of the growing global populations.

Various microorganisms particularly single celled algae such as the marine dinoflagellate Crypthecodinium cohni have been considered candidate organisms. But these algal sources prove to be very expensive because of their low yields and costly extraction procedures. Canola oil, soyabeans, flax seed and certain nuts and seeds (walnut, flax, chia and sometimes pumpkin seed) are a rich source of alpha-linolenic acid, a precursor of DHA. However, the alpha-linolenic acid needs to be converted to DHA and hence is not effective as a supplement to DHA itself.

The production of DHA from Thraustochytrids have been dealt with very exclusively, compared to fish oils they provide a much easy method of production, less fishy smell and highly purified DHA.These group of marine organisms are non-photosynthetic, heterotrophic organisms. But these methods of production mainly include the fermentation and the bioprocessing techniques. But these methods of production mainly include the fermentation and the bioprocessing techniques. However, cost effective alternatives have to be explored for fulfilling the needs of the growing global populations.

The present invention deals with the production of DHA by introduction of genes involved in the biosynthetic pathway of DHA in yeast by recombinant methods. Yeast has long been recognized and used as a host for protein expression since it can offer the processing systems along with the ease of use of microbial systems. As a host, it boasts of a number of benefits as it can be used for the production of both secreted and cytosolic proteins which may require post-translational modifications and its biosynthetic pathway resembles higher eukaryotic cells in many aspects. Moreover, in comparison to the other eukaryotic systems, there is considerably more advanced understanding of its genetics with an ease of manipulation similar to that of E. coli. The expression levels also range to several milligrams per liter of the culture.

### PRIOR ART

The patent No WO2005047485 relates to the filamentous fungal Δ 12 fatty acid desaturases that are able to catalyze the conversion of the oleic acid to linolenic acid (18:2). The Nucleic acid sequence encoding the desaturases, nucleic acid sequences which hybridizes thereto, DNA constructs comprising the desaturase genes, and recombinant host microorganisms expressing increased levels of the desaturases are described. More specifically, the gene encoding a Δ 12 desaturase from the fungus Fusarium moniliforme was isolated, and cloned and efficient conversion of oleic acid to linolenic acid was demonstrated upon expression in an oleaginous yeast.

Another patent published WO2004104167 relates to the invention of Δ 12 fatty acid desaturase able to catalyse the conversion of oleic acid to linolenic acid from Yarrowia lipolytica.

WO2005047480 filed on 12 Nov. 2003 by Yadav, Narendra. S. titled “Cloning and sequencing of fungal Δ15 fatty acid desaturases that are able to catalyse the conversion of linolenic acid to alpha-linoleic acid. Nucleic acid sequences which hybridize thereto, DNA constructs comprising the desaturase genes, and the recombinant host plants and microorganisms expressing increased levels of the desaturases are described. More specifically, the gene encoding the Δ 15 from the fungus Fusarium moniliforme was isolated and cloned, and efficient conversion of LA to ALA was demonstrated upon the expression in oleaginous yeast.

The patent No WO2000040705 describes the identification of the gene involved in the desaturation of polyunsaturated fatty acids at carbon 5 and to uses thereof. The cDNA encoding human Δ 5 desaturases isolated from the human monocyte cDNA library based on its homolog to desaturases from Mortierella alpina are also described.

Patent No WO2000055330 of 18 Mar. 1999 by Napier, Johnathan A (The University of Bristol, UK) titled “Protein and cDNA sequences of Caenorhabditis elegans polyunsaturated fatty acids (PUFA) elongases and their uses thereof.” relates to the cDNA sequences encoding the polyunsaturated fatty acids elongase from Caenorhabditis elegans and also the applications for PUFA elongase. Also reported is the method of synthesizing di-homo-gamma-linolenic acid from gamma-linolenic acid catalyzed by the PUFA elongase enzyme and also the expression of the recombinant PUFA elongase of C.elegans in yeast.

Patent No US 2003163845 relates to the identification of several genes involved in the elongation of polyunsaturated acids (i.e., elongases) and to the uses thereof. It describes the methods of cloning the elongase gene of Mortierella alpina by PCR using primers derived from conserved sequences of the enzyme and adjusted for M.alpina codon usage is demonstrated. Expression of the elongase gene in combination with the Δ5-desaturase genes in Saccharomyces cerevisiae resulted in the appearance of arachidonic acid.

U.S. Pat. No. 6,432,684 relates to the identification of a gene involved in the desaturation of polyunsaturated fatty acids at carbon 5 and to uses thereof. In particular, human Δ 5 was utilized, for example, in the conversion of di-homo-gamma-linoleic acid (DGLA) to arachidonic acid and in the conversion of 20:4n-3 to eicosapentaenoic acid (EPA). The cDNA encoding human Δ5 desaturase was isolated from a human monocyte cDNA library based on its homolog to desaturases from Mortierella alpina desaturase and the use of the Incyte Life seq database of expressed sequence tags are represented.

A recent literature published by Contreras, M. A and Rapoport titled “Recent studies on interactions between n-3 and n-6 polyunsaturated fatty acids in brain and other tissues.” suggests that there is a competition that is mediated between n-3 and n-6 polyunsaturated fatty acids at certain enzymatic steps, particularly those involving polyunsaturated fatty acid elongation and desaturation. One critical enzyme site is delta-6 desaturase. On the other hand, an in vivo method in rats, applied following chronic n-3 nutritional deprivation or chronic administration of lithium, indicates that the cycles of de-esterification/re-esterification of docosahexaenoic acid and arachidonic acid with brain phospholipids operating independently of each other, and thus that the enzymes regulating each of these cycles are not likely sites of n-3/n-6 competition.

The Patent Application DE 2003-10335992 describes the genes for fatty acid elongases and desaturases from variety of taxa for use in the manipulation of patterns of polyunsaturated fatty acid biosynthesis in crop plants or producer organisms. Genes for Δ-6 desaturases, Δ-5 desaturases, Δ-4 desaturases, and Δ-6 elongases are described from organisms including Thalassiosira, Euglena, and Ostreococcus. Omega-3 desaturases from the Pythiaceae and algae including the Prasinophyceae are also described. The construction of a Saccharomyces cerevisiae host expressing genes from Euglena gracilis and Phaeodactylum tricornutum is demonstrated. The organism was able to synthesize docosahexaenoic acid from staeridonic acid or eicosapentaenoic acid.

WO 2002081668 relates to the identification of the genes involved in the desaturation of the polyunsaturated fatty acids at carbon 5, (i.e., “Δ-5-desaturase”) and at carbon 6 (i.e., “Δ6 desaturase” and to the uses thereof. It describes of the use of Δ-5 desaturase for the conversion of di-homo-gamma-linolenic acid (DGLA) to arachidonic acid (AA) an in the conversion of 20:4n-3 to eicosapentaenoic acid (EPA) and the use of Δ-6 desaturase for the conversion of linoleic acid (LA) to g-linolenic acid (GLA). The use of these sequences to identify fatty acid elongase genes of other fungi and mammals is demonstrated.

The patent WO 2001070993 titled “Mammalian Δ6-desaturase genes and promoter regions and screening for compounds modulating enzyme activity or levels” describes the polynucleotides that control desaturase genes and to drug screening assays for identifying pharmaceutically active compounds for use in the treatment of diseases involving abnormal lipid metabolism including diabetic neuropathy, by utilizing fatty acid desaturase enzymes and the genes which encode them as targets for invention. The expression of the gene in Saccharomyces cerevisiae is demonstrated.

There are various limits to the production of DHA in other organisms, which include production of the protein in insoluble forms and high production costs. Saccharomyces cerevisiae offers appealing alternatives that include an extensive toolbox of genetic modification strategies, production of authentic functional products and low culture costs when compared to other expression systems.

Furthermore, large-scale yeast production through fermentative methods and other down stream processes for yeast is simple, safe and well characterized. In addition, yeast is generally considered as a safe organism and owing to their rapid high cell density growth the global demands of Docosahexaenoic Acid can be met easily.

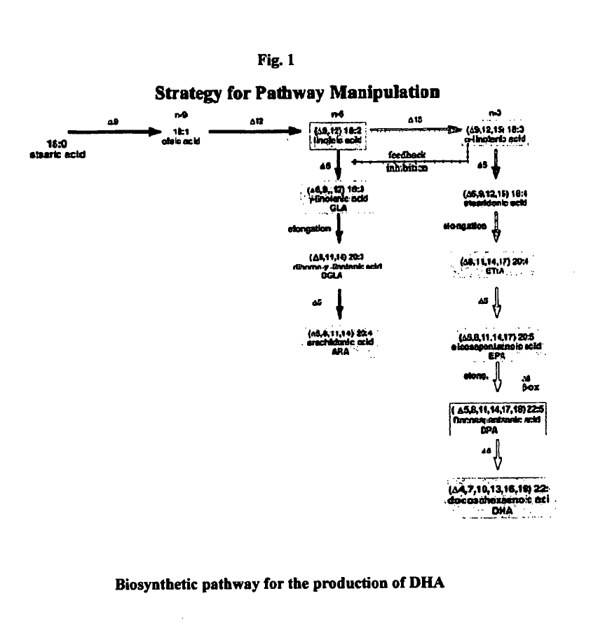

FIG. 1: represents the biosynthetic pathway for the production of DHA

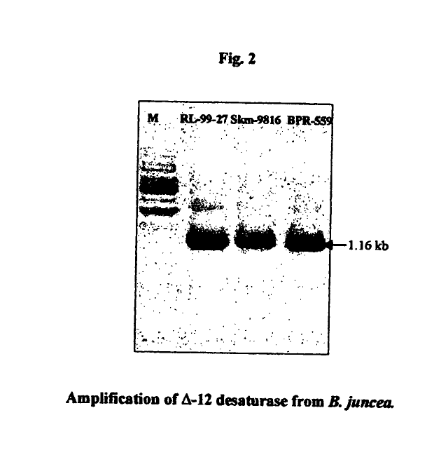

FIG. 2: shows the amplification of Δ 12 desaturase from Brassica juncea

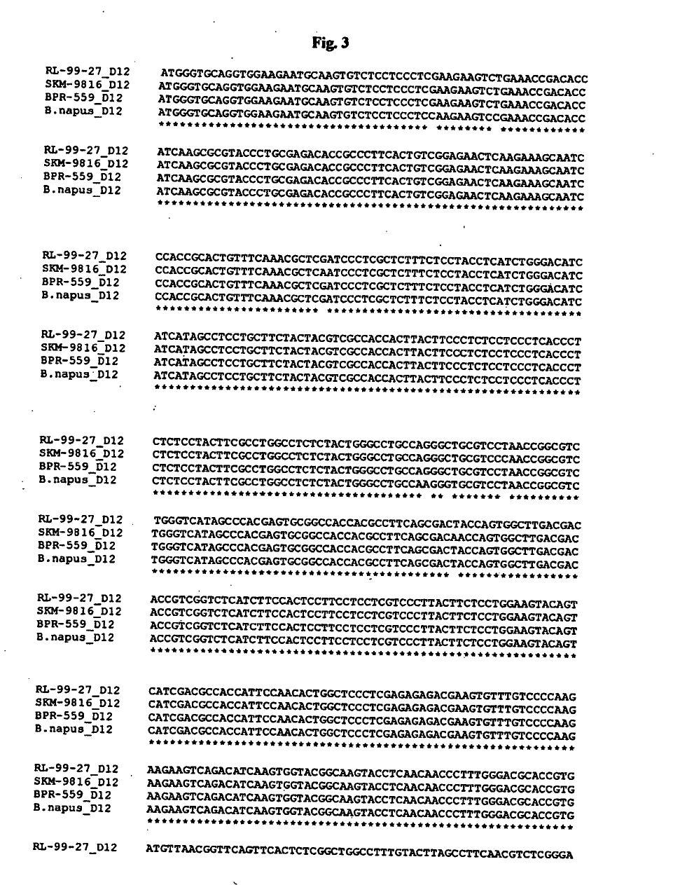

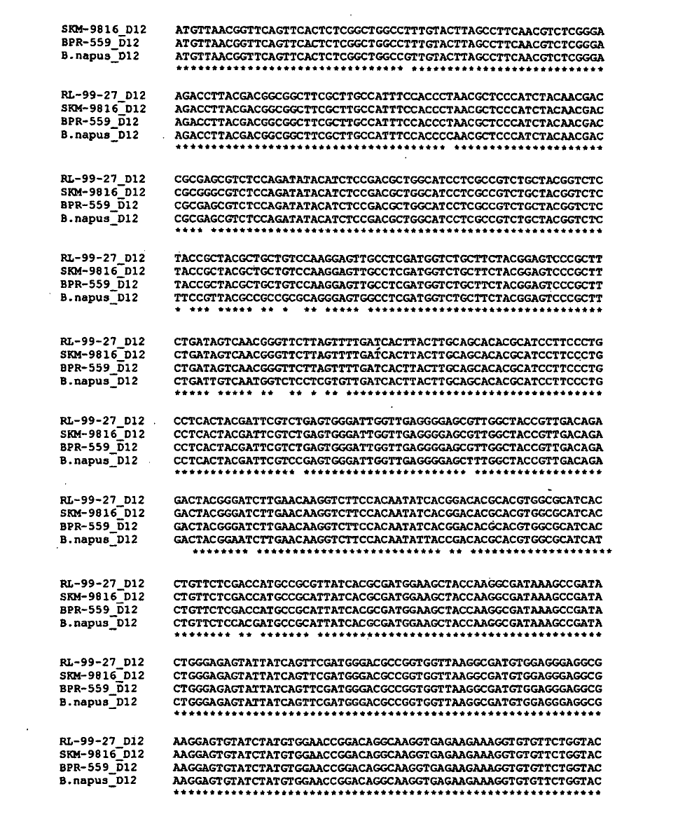

FIG. 3: shows the clustering of the nucleotide sequences of Δ 12 desaturases of RL-99-27, SKM-9816 and BPR-559 with that B.napus.

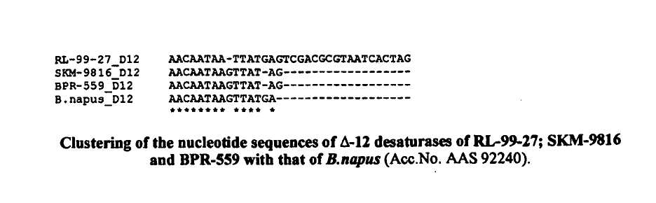

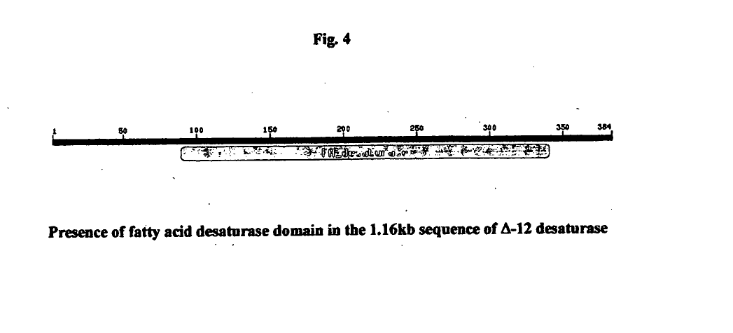

FIG. 4: indicates the presence of fatty acid desaturase domain in the 1.16 Kb Sequence of α 12 desaturase.

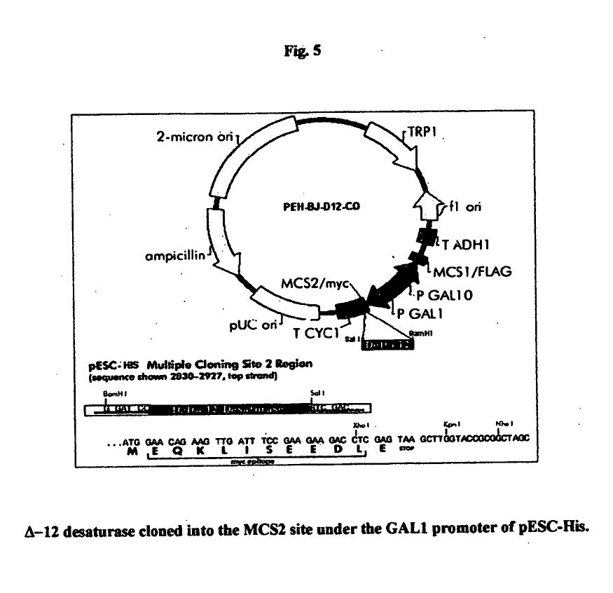

FIG. 5: Δ 12 desaturase cloned into the MCS2 site under the GAL1 promoter of pESC-His.

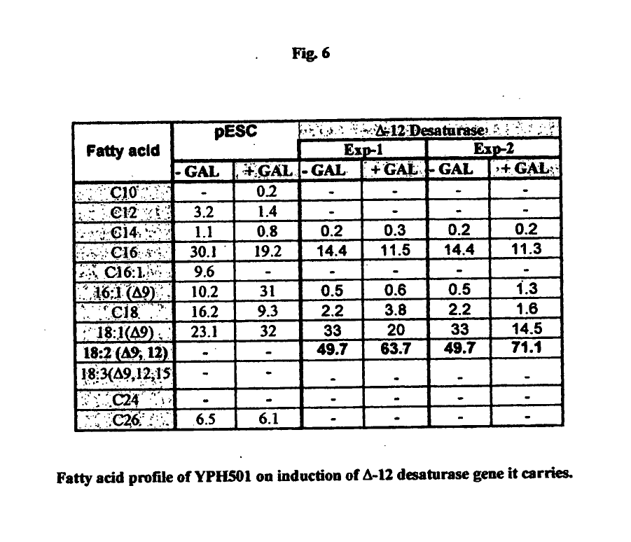

FIG. 6: Fatty acid profile of YPH501 on induction of Δ12-desaturase gene it carries.

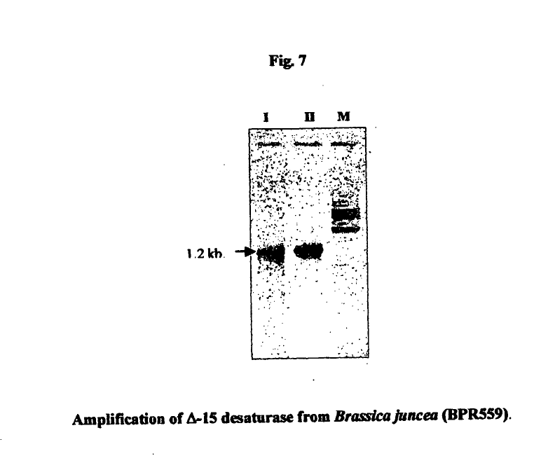

FIG. 7: shows the amplification of Δ15 desaturase from Brassica juncea (BPR559)

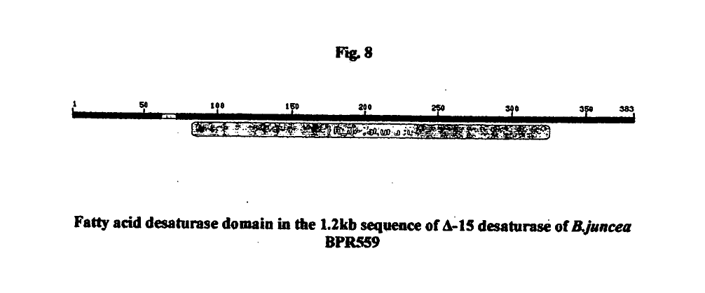

FIG. 8: Fatty acid desaturase domain in the 1.2 kb sequence of Δ15 Desaturase of B.juncea BPR559

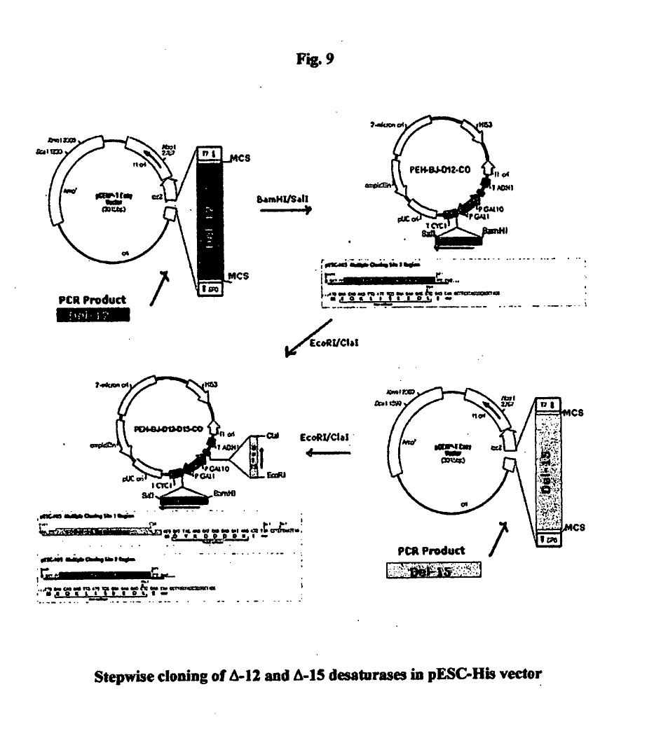

FIG. 9: Representation of the step wise cloning of Δ12 and Δ 15 desaturases In the pESC-His vector.

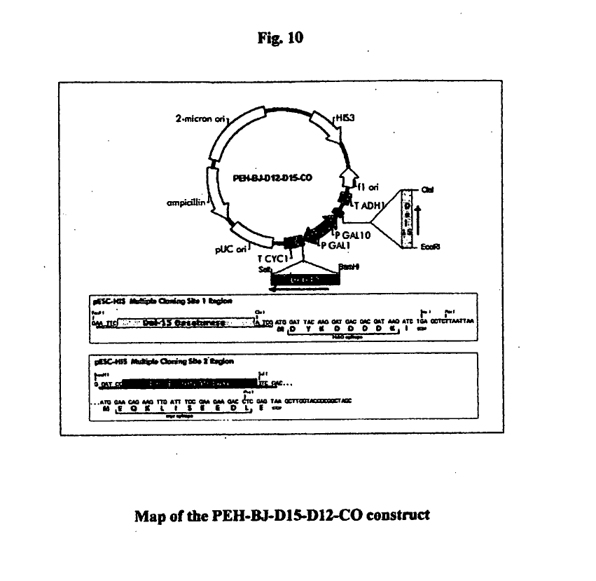

FIG. 10: Map of the PEH-BJ-D15-D12-CO construct

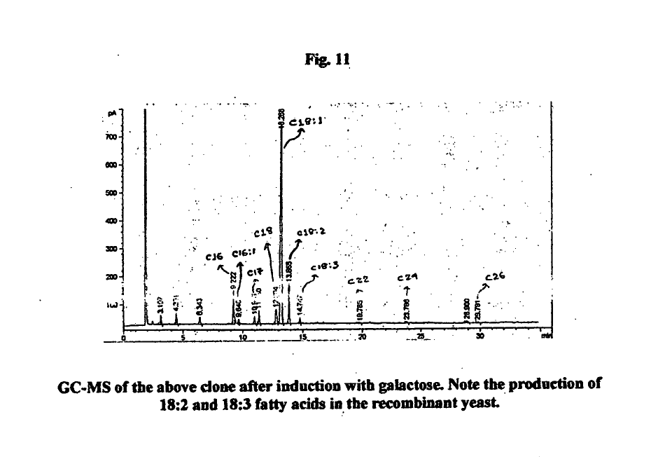

FIG. 11: GC-MS of the above clone after induction with galactose. Indication Of the production of 18:2 and 18:3 fatty acids in recombinant yeast.

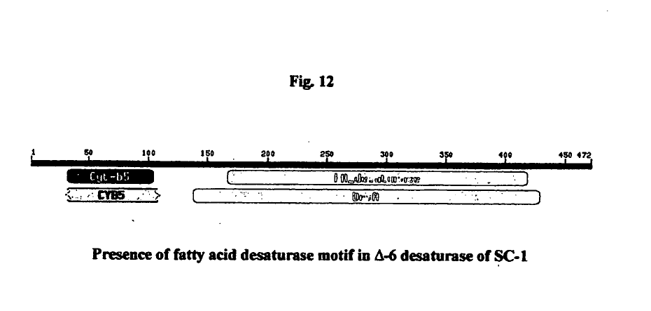

FIG. 12: shows the presence of fatty acid desaturase motif in Δ6 desaturase of SC1

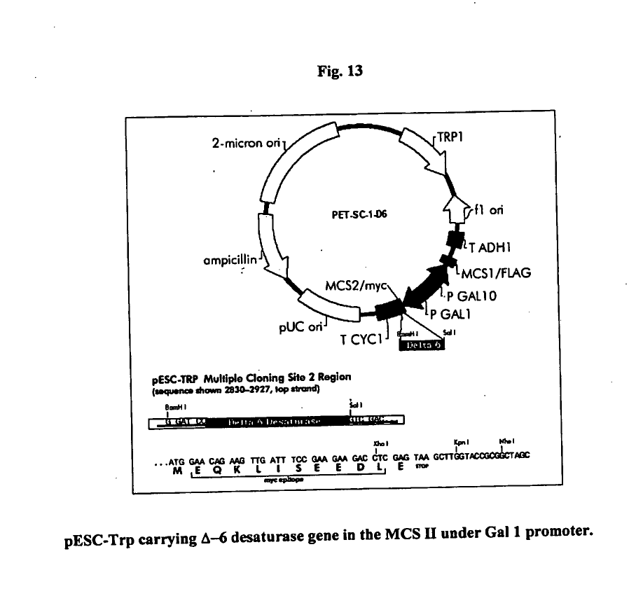

FIG. 13: pESC-Trp carrying Δ6 desaturase gene in the MCS II under GAL 1 Promoter.

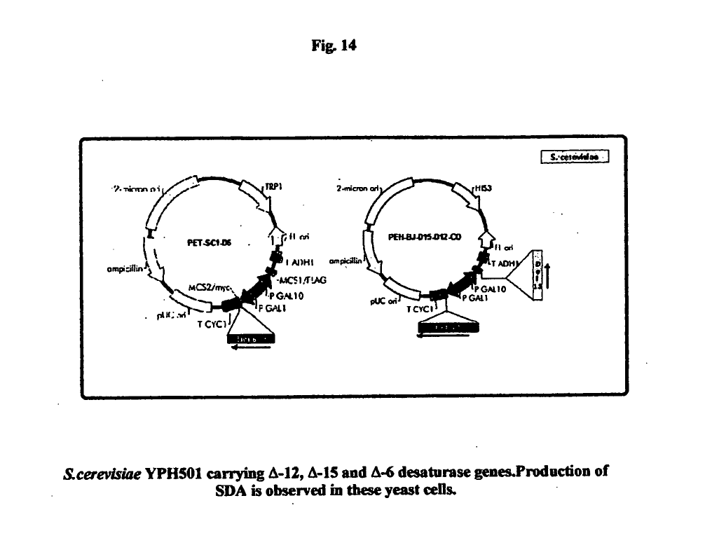

FIG. 14: S.cerevisiae YPH501 carrying Δ-12, Δ 15 and Δ 6 desaturase genes.

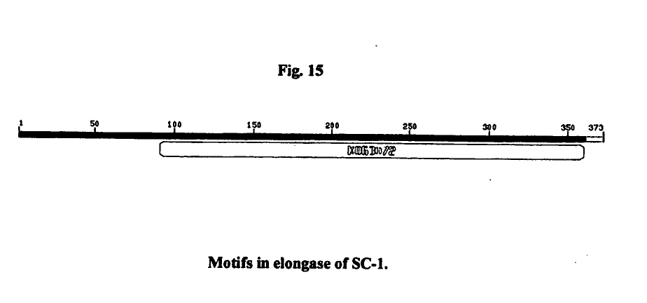

FIG. 15: Motifs in elongase of SC1

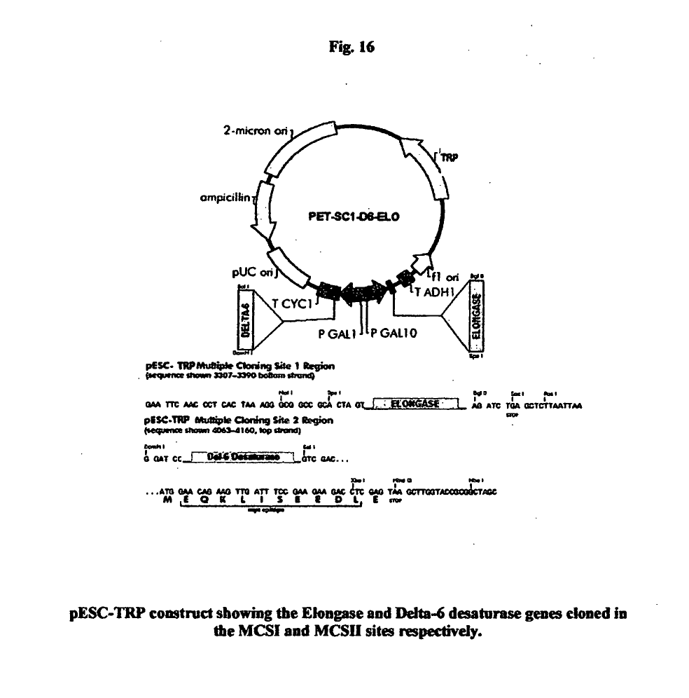

FIG. 16: pESC-TRP construct showing the elongase and Δ6 desaturase genes cloned in the MCSI and MSCII sites respectively.

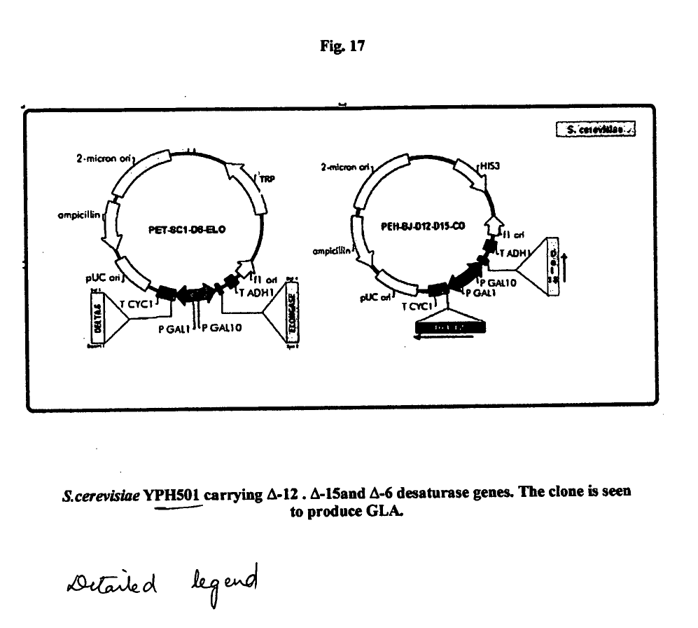

FIG. 17: S.cerevisiae YPH501 carrying Δ12. Δ15 and Δ6 desaturase genes.

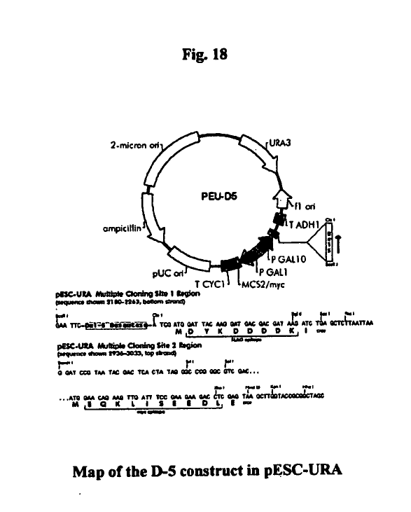

FIG. 18: Map of the Δ5 construct in pESC-URA

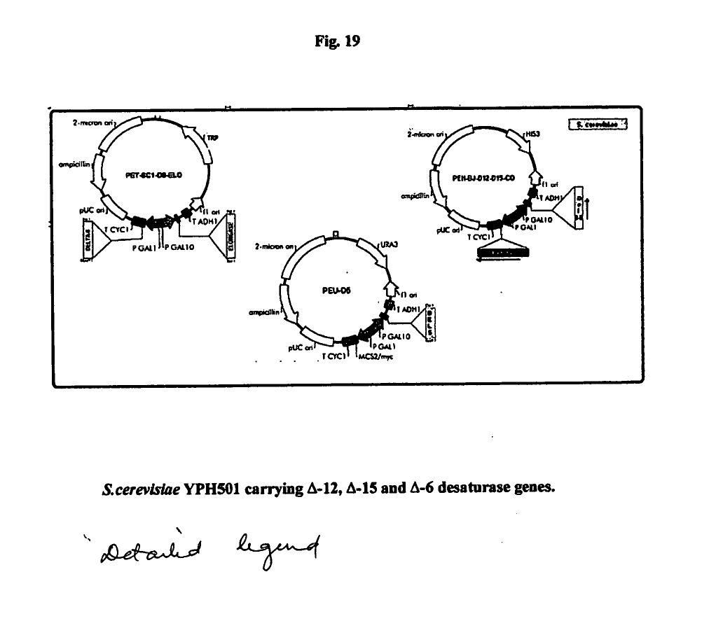

FIG. 19: S.cerevisiae YPH501 carrying Δ 12, Δ15 and Δ 6 desaturase genes.

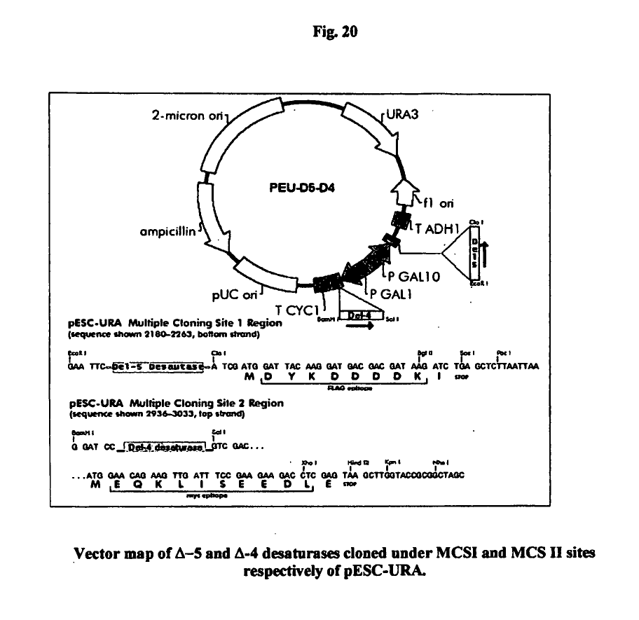

FIG. 20: Vector map of Δ5 and Δ4 desaturases cloned under the MCSI and MCS II sites respectively of pESC-URA

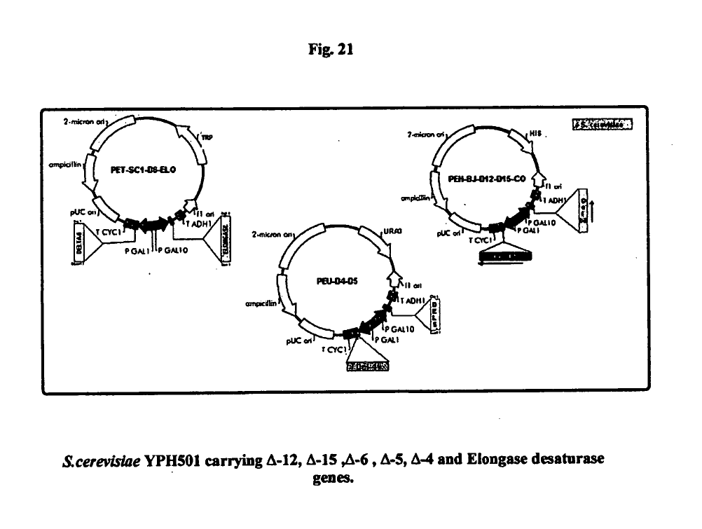

FIG. 21: S.cerevisiae YPH501 carrying the Δ12, Δ 15, Δ6, Δ5, Δ4 and elongase desaturase genes.

### DESCRIPTION OF THE SEQUENCE LISTINGS

SEQ ID NO 1: Sequence of delta12 desaturase from Brassica juncea BPR559 with nucleotide substitutions.

SEQ ID NO 2: Nucleotide Sequence of delta-15 desaturase ORF isolated from Brassica juncea BPR 559

SEQ ID NO 3: Codon optimized sequence of delta-15-desaturase and hence represents an artificial sequence.

SEQ ID NO 4: Full length sequence of delta-6 desaturase of SC1.

SEQ ID NO 5: The nucleotide sequence of delta-6 desaturase codon optimized for introduction into Yeast.

SEQ ID NO 6: Full length elongase sequence.

SEQ ID NO 7: Nucleotide sequence of elongase after codon optimization for introduction into Yeast.

SEQ ID NO 8: Nucleotide sequence of delta-5 desaturase of Phaeodactylum tricornatum.

SEQ ID NO 9: Nucleotide sequence of delta-4 desaturase amplified from Thraustochytrium sp 21685.

### DETAILED DESCRIPTION OF THE INVENTION

DHA is a 22 carbon, 6 double bonds containing polyunsaturated fatty acid that is synthesized from oleic acid through a series of conversions mediated by the desaturases and elongase. This patent describes the pathway engineering of yeast, for the conversion of oleic acid, normally synthesized in yeast, to DHA, to DHA, by introducing five desaturases and elongase isolated from appropriate sources. The steps occurring towards the conversion of oleic acid to DHA is represented in FIG. 1.

The objective of the invention was to isolate the 5 desaturases and the elongase involved in the synthesis of DHA from oleic acid from an appropriate source, clone the genes into the appropriate vectors and introduce them into the yeast for the production of DHA in yeast.

Production of Linoleic acid in Yeast: introduction of Δ12 desaturase

The conversion of the oleic acid to linoleic acid brought about by Δ12 desaturation is the first step in the production of DHA from oleic acid. Linoleic acid undergoes further desaturation and elongation to give rise to highly unsaturated Docosahexaenoic acid. Δ-12 desaturase, the enzyme required for the step has been isolated from three varieties of B.juncea.

Genomic DNA of three varieties of Brassica juncea—RL-99-27, Skm-9816 and BPR-559 were isolated and amplified with primers designed for the amplification of the gene. FIG. 2 describes the amplification of the Δ-12 desaturase from B.juncea. 100 ng of the genomic DNA of RL-99-27, Skm-9816 and BPR-559 varieties of B.juncea were amplified with the primers designed to amplify the ORF of Δ-12 desaturase. M-marker, 1 Kb ladder and the following lanes show the product of amplification of Δ-12 desaturase from the respective varieties as shown in FIG. 2. The amplification of a fragment of the expected size of 1.2 kb.

A fragment of the expected size of 1.2 kb was amplified from all the three varieties of B.juncea. These fragments were cloned into the pGEM^{(T)} Easy vector (Promega). All the three sequences obtained homology to the Δ-12 desaturases of B.napus, B.juncea and B.rapa.

Although the Δ-12 desaturase of B.juncea (all the three varieties) shows homology to the Δ-12 desaturase of various species, its homology to Δ-12 desaturase of B.napus is greater than to that of the other species. The homology of the Δ-12 desaturases isolated from the three varieties to that of B.napus is represented in FIG. 3.

The cDNA sequence of all varieties translates into a protein of 384aa. Search for the motifs confirmed that the sequence isolated had the fatty acid desaturase domain shown in FIG. 4. The above sequence was codon optimized fro yeast and a few of the non conservative aminoacids (as compared to the sequence of B.napus) were replaced with the amino acids of Δ-12 desaturase of B.napus. A total of 23 changes were made to the B.juncea Δ-12 desaturase sequence. The modified sequence of Δ-12 desaturase sequence is represented in Seq ID 1.

Thus, Δ-12 desaturase with the 23 desired nucleotide substitutions has been cloned directionally into the BamHI and SalI sites of pEsc His and the resulting clone named PEH-BJ-D12-CO. The construct was shuttled from E.coli into S.cerevisiae YPH501. Δ-12 desaturase cloned into the MCS site under the GAL1 promoter of pESC-His shown in FIG. 5.

### Proof of Function

All proof of function experiments has been done using PEH-BJ-D12-CO in the yeast strain YPH501. The protocol followed for the experiments is given below.

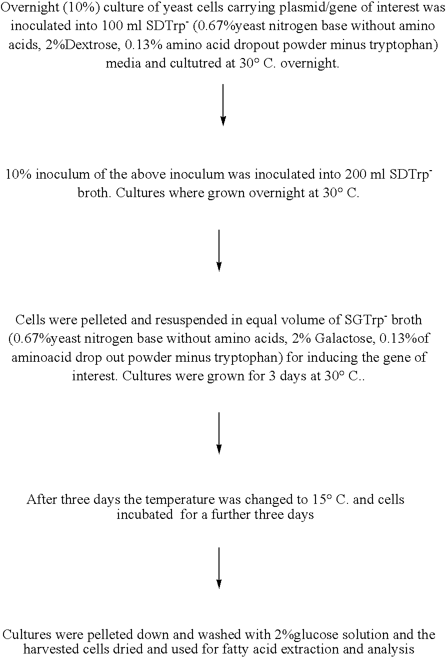

YPH501 cells carrying PEH-BJ-D12-CO were cultured overnight in SD medium at 30° C.; cells were pelleted and resuspended in SG medium the next day. These cells were cultured at 30° C. for 3 days followed by incubation at 15° C. for a further three days (conditions shown to be optimal for the action of the desaturases) (Knutxon et al., 1998). The induced cells were pelleted and subjected to fatty acid analysis. The results of fatty acid analysis are given in FIG. 6.

The experiment have been repeated several times under different conditions and we have observed the occurrence of linoleic acid in the YPH501 cells carrying the Δ-12 desaturase gene. Thus, the Δ-12 desaturase introduced into yeast brings about efficient production of linoleic acid in yeast. In fact, the amount of linoleic acid produced is greater than the amount of oleic acid in the yeast cells. The conversion of oleic acid to linoleic acid in a highly efficient manner probably results in increased production of oleic acid, thus leading to more of linoleic acid being produced. Thus the first step in the pathway engineering of yeast for production of DHA has been successfully accomplished. Production of alpha linolenic acid in yeast

The conversion of linoleic acid to alpha linolenic acid is the next step in the conversion of oleic acid to DHA catalysed by Δ-15 desaturase. This is also the first step in the w-3 pathway. The Δ-15 desaturases are expressed in organisms which produce linolenic acid. In plants the enzyme is expressed in two different tissues—endoplasmic reticulum and chloroplast. The Δ-15 desaturase from the endoplasmic reticulum of B. napus is an 1154bp transcript. The gene is 3.1 kb in length and contains 8 exons; Primers were designed to amplify the ORF of Δ-15 desaturase from the RNA in tissues expressing the gene. B. juncea seeds (BPR559) were treated with 10 μM Abscisic acid for 2 days. Total RNA was isolated from the germinating seedlings and mRNA was prepared from it. The mRNA was reverse transcribed using oligo dT primers. Amplification using 100 ng of the cDNA with specific primers resulted in the amplification of a fragment of the expected size (1.2 kb) represented in FIG. 7.

The 1.2 kb fragment was cloned in pGEM^{(T)}-easy cloning vector and sequenced. The sequence is represented in Seq ID 2.

Motif searches with the sequence confirmed the presence of a fatty acid desaturase domain within the amplified region. It has been represented in the FIG. 8.

The B.juncea sequence has been optimized for expression in yeast. Some of the amino acids were also substituted for improving efficiency of the gene. The resulting sequence is represented in Seq ID 3.

### Cloning of Δ-12 and Δ-15 Desaturase in a Single Construct.

The Δ-12 and Δ-15 desaturases, which constitute the first two steps in the conversion of oleic acid to ALA, have been cloned and proven to function. The codon optimized Δ-12 desaturase and Δ-15 desaturase have been combined together in a single construct. Δ-12 desaturase was cloned into the BamHI and SalI sites of MCSII under the Gal I promoter of pESC-His while Δ-15 desaturase was cloned between the EcoRI and ClaI sites of MCSI under the Gal 10 promoter of the same construct. The stepwise cloning procedure is represented in FIG. 9.

The new construct named PEH-BJ-D15-D12-CO, has been transformed into yeast. The map of the PEH-BJ-D15-D12-CO construct is represented in the FIG. 10.

### Proof of Function:

YPH501 carrying the two codon optimized desaturases have been subject to proof of function experiments as with Δ-12 desaturase. The proof of the production of the 18:2 and 18:3 fatty acids in the recombinant yeast is shown in FIG. 11.

Thus, we have been able to produce ALA in yeast through the introduction of Δ-12 and Δ-15 desaturases into the S.cerevisiae.

Introduction of Δ-6 Desaturase into Yeast

Sequencing of the EST library of SC-1, a thraustochytrid that produces large amounts of DHA resulted in the identification of a Δ-6 desaturase. Screening of the SC-1 BAC library with the above followed by sequencing of the identified BAC clone resulted in the identification of the full length Δ-6 desaturase. The full length sequence of Δ6 desaturase is given in Seq ID 4.

The Δ-6 desaturase sequence was subjected to a motif search for confirmation of the presence of the desaturase domain. The results of motif search of the Δ-6 desaturase from SC-1 is given in FIG. 12.

The above sequence has been codon optimized for expression in yeast. The sequence after the substitution of the codons is shown in the Seq ID No 5.

The optimised Δ-6 desaturase has been cloned into the MCSII site under the Gal1 promoter between BamHI and SalI sites of pESC-Trp (PET-SC1-D6). It has been represented in the FIG. 13.

The construct has been transformed into recombinant yeast carrying Δ-12 and Δ-15 desaturases. S.cerevisiae YPH501 carrying the Δ-12, Δ-15 and Δ-6 desaturase genes is shown in FIG. 14.

Recombinant yeast containing Δ-12, Δ-15 and Δ-6 desaturases were induced by galactose. The production of SDA was observed in these cells.

Introduction of Elongase into Yeast

Elongase has been isolated from the cDNA library of the Thraustochytrid SC—1. The sequence has an ORF of 1119bp, a 5′ UTR of 29 bases and a 3′ UTR of 234 bases. The sequence of the elongase is given in the Seq ID 6.

The sequence shows homology to a number of elongases. Domain prediction using showed the presence of a KOG3072 domain, which is a motif present in most members of the family of elongases. The results of motif search is shown in FIG. 15.

Proof of function of the elongase was conducted wherein fatty acids were extracted from the elongase clone in DH10B before and after induction with IPTG. The extracted fatty acids were esterified and the Fatty Acid Methylesters subjected to GC-MS. Results indicate that the elongase adds 2C to the fatty acids.

The sequence has been codon optimized for expression in yeast and is represented in the Seq ID 7.

Cloning of Elongase and Δ-6 Desaturase in pESC-Trp

Completely codon optimised Elongase and Δ-6 desaturase have been cloned in the pESC-TRP vector in the MCS I and MCS II sites respectively. The vector map showing both the genes in pESC vector is represented in FIG. 16.

The construct has been introduced into the yeast cells carrying the construct ESH-BJ-D15-D12-CO. The construct has been represented in FIG. 17.

The clone called PEHT-12-15-6-Elo has been induced with galactose. This clone is seen to produce Eicosatetraenoic acid.

Production of DPA: Introduction of Δ-5 Desaturase into Yeast.

The next step in the Δ-3 pathway is the conversion of ETA to EPA catalysed by Δ-5 desaturase. The Δ-5 desaturases from P. tricornatum has been cloned and sequenced. The sequence of the desaturases is given in Seq ID 8.

The ORF of these desaturases have been amplified and directionally cloned into MCSI sites between EcoRI and ClaI of pEsc-Ura. The map of the construct is represented in the FIG. 18.

The latter has been shuttled from recombinant yeas carrying Δ-12, Δ-15, Δ-6 desaturases and elongase.

Yeast cells carrying all these five genes have been induced with Galactose. The cells are found to produce DPA. Represented in the FIG. 19.

### Production of DHA in Yeast:

Δ-4 desaturase from Thraustochytrium sps 21685. Has been isolated and cloned. The sequence of the gene is given in the Seq ID 9.

### Cloning of D4 and D5 Desaturases in a Single Construct:

The Δ-4 desaturase has been cloned into the MCS II site of the pESC-URA between Sal I and Bam HI carrying Δ-5 desaturase in its MCSI site between EcoRI and Cla I.

Vector map representation is given in FIG. 20.

S.cerevisiae YPH501 carrying Δ-12, Δ-15, Δ-6, Δ-5, Δ-4 and Elongase desaturase genes represented in the FIG. 21.

The recombinant yeast containing all the six genes of the pathway was induced with galactose. The production of DHA was observed in yeast clones carrying all 6 genes.

1-19. (canceled)

20. A method for producing a polyunsaturated fatty acid wherein said polyunsaturated fatty acid is produced by a recombinant yeast that has been transformed to comprise all the genes involved in the biosynthesis pathway for the production of the fatty acid.

21. The method of claim 20, wherein the yeast is selected from the group consisting of Saccharomyces cerevisiae and other oleaginous species.

22. The method according to claim 20, wherein said yeast is transformed with SEQ ID NO:1 which encodes the delta-12 desaturase enzyme.

23. The method according to claim 20, wherein SEQ ID NO:1 is introduced into a yeast vector which is used for transforming the yeast.

24. The method according to claim 20, wherein said polyunsaturated fatty acid is linoleic acid.

25. The method according to claim 20, wherein said yeast is transformed with a nucleic acid sequence having SEQ ID NO:3 encoding the delta-15 desaturase enzyme.

26. The method according to claim 25, wherein SEQ ID NO:3 is cloned into a yeast vector comprising SEQ ID NO:1 to form a single construct carrying polynucleotide sequences that encode delta-15 and delta-12 desaturases.

27. A method for producing alpha-linolenic acid comprising the steps of:
a. isolating a nucleic acid sequence comprising, or complementary to, at least 50% of a nucleotide sequence selected from the group consisting of SEQ ID NO:1 and SEQ ID NO:3;
b. constructing a vector comprising the said isolated nucleotide sequence of step (a); and
c. introducing said vector of step (b) into a host yeast cell for a time and under conditions sufficient for the expression of alpha-linolenic acid encoded by said nucleotide sequence of step (a).

28. The method according to claim 20, wherein the yeast is transformed with SEQ ID NO:5, which encodes delta-6 desaturase.

29. The method according to claim 28, wherein SEQ ID NO:5 is introduced into a yeast vector which is used for transforming a yeast host cell thereby conferring the host cell the ability to express steridonic acid.

30. The method according to claim 20, wherein the yeast is transformed with SEQ ID NO:7, which encodes an elongase enzyme.

31. The method according to claim 30, wherein SEQ ID NO:7 is introduced into a yeast vector comprising SEQ ID NO:5 to form a single construct carrying the nucleic acid sequences of SEQ ID NO:5 and SEQ ID NO:7.

32. A method for producing eicosatetranoic acid comprising the steps of:
a. isolating a nucleotide sequence comprising, or complementary to, at least 50% of a nucleotide selected from the group consisting of SEQ ID NO:5 and SEQ ID NO:7;
b. constructing a vector comprising the said isolated nucleotide sequence of step (a); and
c. introducing the said vector of step (b) into a host yeast cell for a time and under conditions sufficient for the production of eicosatetranoic acid encoded by the said nucleotide sequence of step (a).

33. The method according to claim 20, wherein the yeast is transformed with SEQ ID NO:8 which encodes the enzyme delta-4 desaturase.

34. The method according to claim 20, wherein SEQ ID NO:8 is introduced into a yeast vector which is then used for transforming the yeast.

35. The method, according to claim 34, wherein said vector further comprises SEQ ID NO:9.

36. A method of producing docosahexaenoic acid comprising the steps of:
a. isolating a nucleic acid sequence comprising, or complementary to, at least 50% of the nucleotide selected from the group consisting of SEQ ID NO:8 and SEQ ID NO:9;
b. constructing a vector comprising the said isolated sequence of step (a); and
c. introducing the said vector of step (b) into a host yeast cell, for a time and under conditions sufficient for the expression of docosahexaenoic acid encoded by the said nucleotide sequence of step (a).

37. A host yeast cell transformed to comprise polynucleotides that encode the enzymes required for the production of a polyunsaturated fatty acid that is not produced in the wild type of said host cell.

38. The host cell of claim 37, wherein the expression of the nucleotide sequences results in the production of docosahexaenoic acid.

39. The host cell according to claim 37, wherein said cell is Saccharomyces cerevisiae or another oleaginous species.

40. The method, according to claim 20, wherein the fatty acid that is produced is docosahexaenoic acid.
